(19) United States
(12) Patent Application Publication
(10) Pub. No.: US 2007/0270934 A1
(43) Pub. Date: Nov. 22, 2007

(54) Sensor, delivery system, and method of fixation

(76) Inventors: David Stern, Grayson, GA (US); Jason White, Decatur, GA (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/724,611

(22) Filed: Mar. 14, 2007

Related U.S. Application Data
(60) Provisional application No. 60/782,314, filed on Mar. 14, 2006.

(51) Int. Cl. A61F 2/06

(52) U.S. Cl. 623/1.11

(57) ABSTRACT

An implant assembly for releasing into a vessel at an implant location includes an intracorporeal device and an anchor. The anchor comprises a pair of resiliently deformable loops operatively associated with the intracorporeal device, both of the loops extending toward the same side of a plane defined by the intracorporeal device. The deformable loops have a relaxed state and a deformed state. When the deformable loops are in a relaxed state, the implant assembly has a major dimension in a direction out of the plane that is greater than the diameter of a vessel at the implant location. The loops are deformable to permit insertion of the implant assembly into the vessel. The tendency of the loops to return to their relaxed state exerts a force on a wall of the vessel that imposes the intracorporeal device against an opposite wall of the vessel.

### RELATED APPLICATION DATA

This application claims priority to provisional U.S. Application No. 60/782,314, filed Mar. 14, 2006.

### BACKGROUND OF THE INVENTION

1. Field of the Invention

This invention relates generally to implantation of intracorporeal devices into vessels and to fixing the devices, either permanently or temporarily, within the vessel.

2. Description of Related Art

In recent years, the long-sought goal of implantable biosensors has begun to see realization and, in some cases, clinical use. As this concept has seen continued research and development, issues regarding intracorporeal fixation of the sensors have come to light. Particularly within blood vessels, the sensor is subjected to a continuous, pulsatile flow. Blood vessels are thus a difficult environment in which to secure a sensor or other apparatus reliably without unduly restricting blood flow or impairing the vessel wall.

One major vessel of interest in the realm of cardiology is the radial artery, which runs distally down the anterior part of the forearm. The radial artery is a particularly challenging location in which to secure an intracorporeal device because, in addition to the above considerations, the vessel is small, displays variability with regards to the net force resulting from blood flow, and progressively widens in the direction of blood flow.

There are a number of design considerations for an ideal fixation device intended for intravascular fixation at such locations. The anchoring structure should be active and orient the sensing surface of the sensor towards the flow lumen to maintain blood flow past the sensor. The anchor structure should minimize the area of contact and forces exerted on the vessel wall while possessing sufficient contact area and force to fix the implant assembly securely at the implant site. Preferably, the anchoring structure will position the sensor such that it lies parallel to and flush with the vessel wall. The sensor and anchoring structure combination that comprise the implant assembly should not occupy so much of the cross-sectional area of the lumen that blood flow is restricted. The implant assembly should be amenable to delivery through low-profile catheters, preferably six French or less. Furthermore, the anchoring structure should be designed so that it is possible to deploy the device reliably with a user-selected orientation. Finally, the anchoring structure should be sufficiently versatile as not to depend, within physiologically relevant ranges, on the size of the vessel at the intended implant site in order to maintain its position.

There have been attempts to create devices intended to hold intracorporeal devices fixed within vessels. However, these attempts fall short of meeting all of the necessary requirements outlined above. Such devices include a self-expansible stent on which an intracorporeal device is mounted. This stent maintains a known length when implanted in a vessel where only the approximate diameter can be determined. Other devices and methods include fixation of a sensor in a bodily lumen, in which the sensor support is coupled to a fixation device. The fixation device is a stent or ring, has a sensor support coupled thereto and is intended to be sutured to the vessel wall or held in place by plastically deforming the structure using a balloon catheter. The ring is essentially a stent with an abbreviated length and suffers from the same shortcomings as traditional stent devices.

A stent is designed with mechanical characteristics that enable it to hold open diseased vessels post dilation. Therefore, the radial strength of the stent is greater than the inward radial forces exerted during vessel recoil. This primary requirement leads to a mismatch in compliance, with that of the stent dominating. Subsequently, stress concentrations are created at the interface of the stent and vessel. These stress concentrations are greatest at the terminal ends of the stent, where there is an abrupt transition in stiffness between the stented and unstented segments of the vessel. Because undiseased vessels are usually more compliant compared to diseased ones, this compliance mismatch is amplified when placing a stent in healthy vasculature. Along similar lines, accurate stent sizing in the vessel is critical, especially in the case of the pulmonary artery. Accurate stent sizing to prevent migration and to avoid perforation of the vessel wall could be more difficult in healthy vasculature. Thus, the physician must be conscious of the particulars of vessel compliance along with stent recoil and radial strength to choose the stent whose expanded diameter is best for a given vessel. This determination presents its own set of challenges and requires an undesirable increase in complexity, e.g., in deployment and risk of complication. Therefore, the use of a stent to maintain an intracorporeal device in a vessel is not optimal.

Thus, a need exists for devices and methods for fixing intracorporeal devices which satisfy the design requirements described herein. Furthermore, a need exists to deliver and fix such devices in a safe, simple and predictable manner.

### SUMMARY OF THE INVENTION

Stated generally, the present invention relates to an apparatus and method of deployment and fixation of an implant assembly. In one aspect of the invention the deployment is achieved by using a delivery apparatus to deliver an intracorporeal device to a deployment site. In another aspect of the invention, fixation of the device is accomplished by using an anchoring structure. In one embodiment, the anchoring structure anchors the intracorporeal device at a set location and against the vessel wall. In certain embodiments, the intracorporeal device may be either a wired or a wireless device.

Thus there is a need to provide an implant assembly having an anchoring structure for fixation within a vessel.

There is a further need to provide an implant assembly adapted to be delivered via a delivery apparatus, such as a catheter.

There is still a further need to provide an intracorporeal device that does not obstruct the flow of blood through a vessel.

There is yet a further need to provide a sensor that may be delivered and oriented in a vessel so that the sensing surface faces the flow of blood through the lumen.

Other objects, features, and advantages of the present invention will become apparent upon reading the following specification, when taken in conjunction with the drawings and the appended claims.

### BRIEF DESCRIPTION OF THE DRAWINGS

FIG. 1 is an isometric view of a first embodiment of an implant assembly with the anchoring structure in a relaxed state.

FIG. 2 is a cutaway view of a vessel showing the implant assembly of FIG. 1 fixed therein in a deployed state.

FIG. 3 is a side cross-sectional view of an apparatus for delivery of the implant assembly of FIG. 1 to a target location within a vessel.

FIGS. 4 and 5 illustrate delivery of the implant assembly of FIG. 1.

FIG. 6 is an isometric view of a second embodiment of an implant assembly of this invention with the anchoring structure in a relaxed state.

FIG. 7 is an isometric view of a third embodiment of an implant assembly of this invention with the anchoring structure in a relaxed state.

### DETAILED DESCRIPTION OF THE DISCLOSED EMBODIMENTS

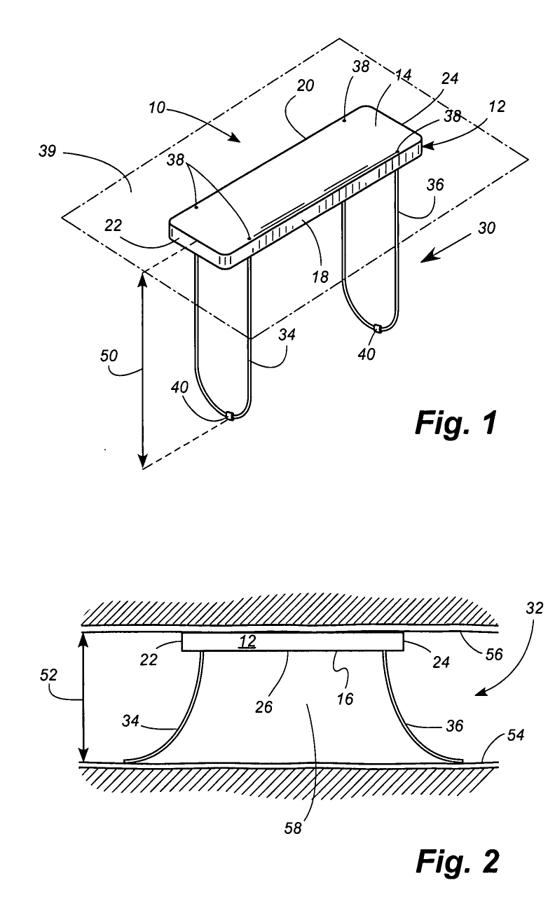

Referring now to the drawings, in which like numerals indicate like elements throughout the several views, FIGS. 1 and 2 illustrate an implant assembly 10 comprising an intracorporeal device 12. The phrase “intracorporeal device” as used in this document includes any device implantable within the body of a patient. Such devices can include, e.g., sensors that measure chemical and/or physical parameters, devices configured to perform a function, e.g. drug delivery devices, or other similar devices. The intracorporeal device may communicate with external electronics, either wirelessly or by being placed in physical contact with the external electronics, such as by a lead wire.

The intracorporeal device is generally rectangular and comprises an upper wall 14, a lower wall 16 (FIG. 2), first and second side walls 18, 20, and first and second end walls 22, 24. The intracorporeal device 12 of the disclosed embodiment is a pressure sensor. Toward that end, the lower wall 16 comprises a deflectable region 26 (FIG. 2) that deflects in response to a physiologically relevant range of pressures.

The intracorporeal device 12 of the implant assembly 10 has a width of about 0.5 to about 4 mm, a height of about 0.5 to about 4 mm, and a length of about 0.5 to about 12 mm. In one embodiment, the intracorporeal device has a width of 2 mm, a height of 0.4 mm, and a length of 10 mm. In the disclosed embodiment, the intracorporeal device 12 comprises a circuit having at least one component that is coupled to the deflectable region 26. The circuit has a characteristic impedance that changes as the deflectable region 26 moves. The external electronics detects this impedance and converts it to a pressure. Examples of such devices are disclosed in commonly owned patents U.S. Pat. Nos. 6,855,115 and 7,147,604; and in co-pending, commonly owned applications Ser. Nos. 10/054,671, 10/886,829, 10/215,377, and 10/943,772, incorporated herein by reference.

The implant assembly 10 further comprises an anchoring structure 30 used to stabilize the intracorporeal device 12 within the body, for example, within a blood vessel 32 (FIG. 2). The anchoring structure 30 includes first and second loops 34, 36. Each loop 34, 36 has two ends 38, each of which are attached to the intracorporeal device 12. The loops 34, 36 both project away from the intracorporeal device 12 on the same side of a plane 39 defined by the longitudinal and lateral axes of the intracorporeal device.

Optionally, the loops 34, 36 include a radiopaque feature 40. The radiopaque feature 40 may be a metal and, in one example, includes Pt/Ir tubing segments crimped to the wire loops 34, 36.

The intracorporeal device 12 includes a coating. In the implant assembly 10, the anchoring structure 30 is affixed to the intracorporeal device 12 by inserting the wires through the coating. However, similar results can be achieved by constructing the intracorporeal device 12 of a polymeric material, in which case the anchoring structures could be affixed to the intracorporeal device by threading the wires directly through the polymeric material comprising the device. Materials used in the construction of such intracorporeal devices or coatings could be any biocompatible polymer, including but not limited to biocompatible silicone rubber, FEP, PTFE, urethane, PVC, nylon, and polyethylene.

The anchoring structure 30 of the implant assembly 10 is manufactured by bending two wires to form the loops 34, 36. Each end 38 of the loops 34, 36 is inserted into corresponding holes in the intracorporeal device 12. In the implant assembly 10, the anchoring structure 30 is formed from metal or polymer and is in the form of a wire structure. In the disclosed embodiment 10, the wire diameter of the anchoring structure 30 is in the range of about 0.001 to about 0.015 inches. The material comprising the wire can be any resiliently deformable biocompatible material known in the art that possesses suitable material properties to be useful for the purpose at hand. The material comprising the wire can be a polymer or a metal, such as nitinol, stainless steel, eligiloy, cobalt chrome alloys, or any other suitable metal or alloys thereof. In a further embodiment, if the wire is comprised of a metal material, the biocompatible wire is coated with a dielectric material, such as, but not limited to, PTFE, polyurethane, parylene and diamond-like carbon (DLC), such that when the intracorporeal device 12 comprises an RF sensor, the material will not electromagnetically interference with the function of the intracorporeal device.

The anchoring structure 30 of the implant assembly 10 has a relaxed state and a deployed state. In the relaxed state, shown in FIG. 1, the height 50 (FIG. 1) of the implant assembly is greater than the diameter 52 (FIG. 2) of the vessel 32 at the implant site. In the deployed state, the anchoring structure 30 elastically deflects so that the anchoring structure exerts radial force on the lower wall 54 of the vessel 32. This force imposes the upper wall 14 of the intracorporeal device 12 firmly against the upper wall 56 of the vessel 32. In this orientation, the deflectable region 26 of the intracorporeal device 12 is directed toward the lumen 58 of the vessel to facilitate pressure measurement. The wire members of the anchoring structure 30 act as springs to ensure that the implant assembly 10 maintains its position within the vessel 32 while minimizing the force and contact area between the anchoring structure 30 and the vessel wall 54.

The implant assembly 10 obstructs approximately 50% or less of the cross-sectional area of the vessel 32 within which it resides. Preferably, the implant assembly 10 obstructs 20% or less of the cross-sectional area of the vessel 32. Minimizing the obstruction of flow within the vessel 32 allows the intracorporeal device 10 to remain secured in position within the vessel without significantly impacting the flow within the vessel.

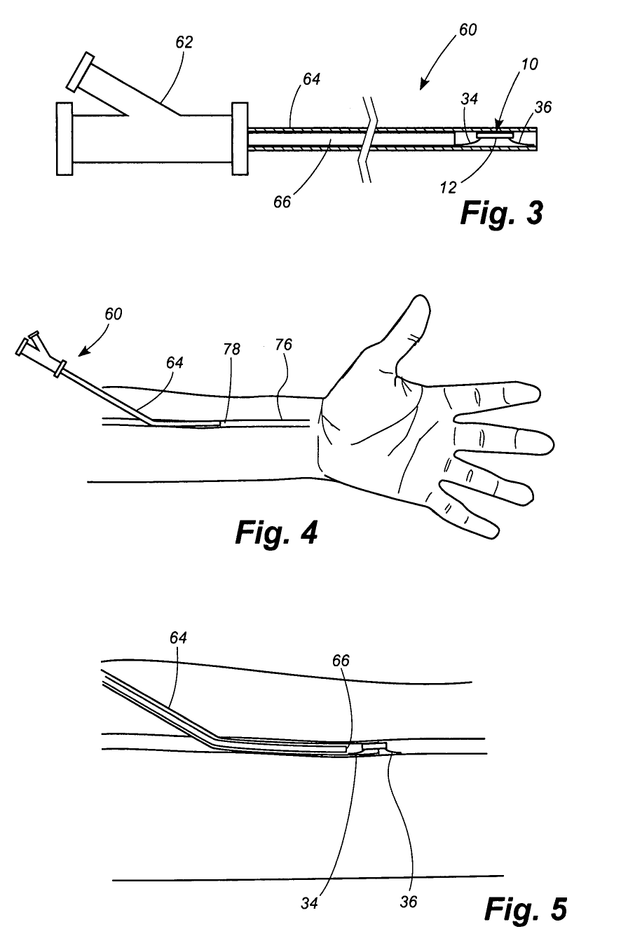

Implant assembly units of this invention may be delivered to the implant site using a delivery apparatus 60 of the type shown in FIG. 3. The delivery apparatus 60 includes a Tuohy Borst Y-connector 62 having a catheter 64 attached to its proximal end. A pusher rod 66 is slidably positioned within the lumen of the catheter 64. With the implant assembly 10 loaded into the distal end of the catheter 64, the legs 32, 34 extend outward and away from the intracorporeal device 12.

Delivery of an implant assembly 10 to a radial artery 76 is illustrated in FIGS. 4 and 5. Access to the radial artery 76 proximal to the intended delivery site 78 is obtained through standard techniques. The distal end of a 6 French by 13 cm delivery apparatus 60 is introduced into the radial artery 76 using standard technique. The distal end of the delivery apparatus 60 is advanced until the implant assembly 10 is located at the intended site 78 of delivery. The optional radiopaque features 40 (FIG. 1) provided on the implant assembly 10 aid in positioning the delivery apparatus 60 when viewed on a fluoroscope. The delivery apparatus 60 can be rotated about its longitudinal axis to provide a correct delivery orientation. Optionally, a torquable delivery catheter shaft can be provided when lateral orientation is important in the operation of the sensor. The pusher rod 66 is then held in place to maintain the position of the implant assembly 10 while the delivery apparatus 60 is retracted in the proximal direction. Once the implant assembly 10 is deployed, the pusher rod 66 is withdrawn from the vessel 76. Then, as shown in FIG. 2, the position of the implant assembly 10 is maintained by the forces created by the spring-like loops 34, 36 comprising the anchor structure of the intracorporeal device.

The delivery device and methods described herein may be modified to provide for delivery of the implant assemblies of the present invention to a variety of implant sites. Delivery sites include, but are not limited to, the radial and brachial arteries.

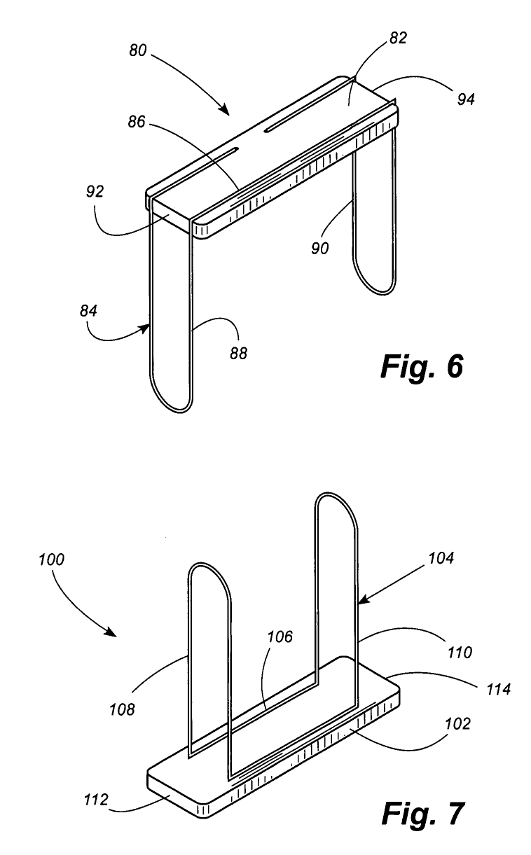

FIG. 6 illustrates an alternative embodiment of an implant assembly 80. The implant assembly 80 includes an intracorporeal device 82 and an anchoring structure 84. The anchoring structure 84 comprises a single wire 86 forming first and second loops 88, 90. The loops 88, 90 extend from opposite ends 92, 94 of the intracorporeal device 82. The wire 86 can be bonded to the upper surface of the intracorporeal device 82, embedded within the material forming the intracorporeal device, or embedded within a coating applied to the intracorporeal device.

In still another embodiment, illustrated in FIG. 7, an implant assembly 100 includes an intracorporeal device 102 and an anchoring structure 104. The implant assembly 100 is shown inverted as compared to the implant assemblies 10, 80, to illustrate the attachment of the anchoring structure 104 to the bottom surface of the intracorporeal device 102. The anchoring structure 104 comprises a single wire 106 forming first and second loops 108, 110. The loops 108, 110 extend from points interior of the ends 112, 114 of the intracorporeal device 102. The wire 106 can be bonded to the lower surface of the intracorporeal device 102, embedded within the material forming the intracorporeal device, or embedded within a coating applied to the intracorporeal device.

The implant assemblies disclosed herein rely on the physical size of the expanded anchoring structure coupled with the spring constant of the wire used to provide an anchoring structure suitable for preventing further distal movement and for minimizing the area and force between the implant assembly and the vessel wall. This concept is contrary to stent or vena cava filter type mechanisms, wherein fixation is achieved by radially exerted force over a substantially greater area of interface and/or by hook or barb attachment features.

Unless otherwise stated, terms used herein such as “top,” “bottom,” “upper,” “lower,” “left,” “right,” “front,” “back,” “proximal,” “distal,” and the like are used only for convenience of description and are not intended to limit the invention to any particular orientation. Similarly, unless specifically claimed, where dimensions of components are given, such dimensions are for purposes of example only and are not intended to limit the scope of the invention.

Finally, it will be understood that the preferred embodiments have been disclosed by way of example, and that other modifications may occur to those skilled in the art without departing from the scope and spirit of the appended claims.

What is claimed is:

1. An implant assembly for releasing into a vessel at an implant location having a diameter, the implant assembly comprising:
an intracorporeal device comprising a longitudinal axis and a lateral axis, said longitudinal and lateral axes defining a plane; and
an anchor comprising a pair of resiliently deformable loops operatively associated with the intracorporeal device, both of said loops extending toward the same side of said plane;
wherein said deformable loops have a relaxed state and a deformed state;
wherein when said deformable loops are in a relaxed state, the implant assembly has a major dimension in a direction out of said plane that is greater than the diameter of a vessel at an implant location; and
wherein said loops are deformable so as to permit insertion of said implant assembly into said vessel, the tendency of said loops to return to their relaxed state exerting a force on a wall of said vessel that imposes said intracorporeal device against an opposite wall of said vessel.

2. The implant assembly of claim 1, wherein the intracorporeal device comprises a pressure sensor.

3. The implant assembly of claim 2, wherein said pressure sensor comprises a deflectable wall portion.

4. The implant assembly of claim 3, wherein said deflectable wall portion is oriented toward said same side of said plane as said loops.

5. The implant assembly of claim 1, wherein said loops are formed from separate wires.

6. The implant assembly of claim 5, wherein said separate wires have ends, and wherein said ends of said wires are anchored to said intracorporeal device.

7. The implant assembly of claim 1, wherein said loops are formed from a single wire.

8. The implant assembly of claim 7, wherein the intracorporeal device comprises a coating, and wherein portions of said wire are inserted underneath the coating of the intracorporeal device to attach the loops to the device.

9. The implant assembly of claim 7, wherein the intracorporeal device is at least partially formed from a material, and wherein portions of said wire are embedded within the material that at least partially forms the intracorporeal device to attach the loops to the device.

10. The implant assembly of claim 7, wherein said wire is anchored to a surface of said intracorporeal device that is on the opposite side of said plane from said loops.

11. The implant assembly of claim 7, wherein said wire is anchored to a surface of said intracorporeal device that is on the same side of said plane from said loops.

12. The implant assembly of claim 1, wherein the implant assembly is at least partially radiopaque.

13. A method of deploying and fixing an implant assembly in a vessel, comprising:
(a) mounting an implant assembly on a delivery apparatus, the implant assembly comprising an intracorporeal device and an anchor, the delivery apparatus comprising means for retaining the implant assembly on the catheter;
(b) placing a vessel introducer in an access site;
(c) placing the catheter and implant assembly into the vessel introducer;
(d) navigating the catheter to a deployment site, the deployment site having a diameter;
(e) actuating the implant assembly retaining means to disengage the implant assembly;
(f) removing the catheter from the body;
(g) allowing the anchor structure of the implant assembly to expand to hold the implant assembly against a wall of the vessel.

14. The method of claim 13, further comprising rotating the catheter to orient the implant assembly in a desired direction after navigating the catheter to a deployment site.
